SEQUENCE LISTING (1 sequence)

SEQ ID NO: 1 - DNA, 658 bp, Homo sapiens
  repeat_region (285)..(344)
ctaaatgtacatttaaagagggtgtgaggacgcaagcagtcagcagaggattccagcaggtgacattttagggagctggagacagcagagcctggggttgctaagttcctgatgttgcccaccaggctattgctctgagcagcgctgcctcccagctttctggaaccttctgggacgcctggggtgcatcaagtcccaaggggacagggagcagaagggggggctctggaaggagcaaaatcacacccagagcctgcagcttctcagatttccttaaaggttttgtgtgtgtgtgtgtgtgtgtgtgtgtgtgtatgtgtgtgtgtgtgtgtgtgtgtgtgtgttttctctaaaagtcctatggccagactttgtttcccaagggtcatatgactgctcctctccaccccacactggcccggggcgggctgggcgcgggccctgcgggtgttgcaacgcccggccagaaagtgggcatcagctgttccgcctggcccacgtgacccgccgagcataaatgtgaccggccgcggctccggcagtcaacgcctgcctcctctcgagcgtcctcagcgcagccgccgcccgcggagccagcacgaacgagcccagcaccggccggatggagcgtccgcaacccgacaggcaagcgcggggc

(19) United States
(12) Patent Application Publication
(10) Pub. No.: US 2005/0026186 A1
(43) Pub. Date: Feb. 3, 2005

(54) Method for evaluating an inclination of a subject to lung cancer

(76) Inventors: Mutsuo Yamaya, Sendai-shi (JP); Hiroshi Kubo, Sendai-shi (JP); Satoshi Suzuki, Sendai-shi (JP); Hidetada Sasaki, Sendai-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/855,427

(22) Filed: May 28, 2004

(30) Foreign Application Priority Data
Jun. 12, 2003 (JP) 2003-168012

(51) Int. Cl. C12Q 1/68

(52) U.S. Cl. 435/6

(57) ABSTRACT

The present invention provides a method for evaluating an inclination of a subject to lung cancer, the method comprising preparing a DNA, from the sample, containing at least one allele which comprises a GT repeat located upstream to a heme oxygenase-1 gene, and determining the repeat number of the GT repeat in each allele in the sample wherein the presence of at least one allele comprising a GT repeat whose repeat number is not less than 33 being an indication for an inclination of a subject to lung cancer. In addition, the present invention provides a kit for evaluating an inclination of a subject to lung cancer, by means of the method.

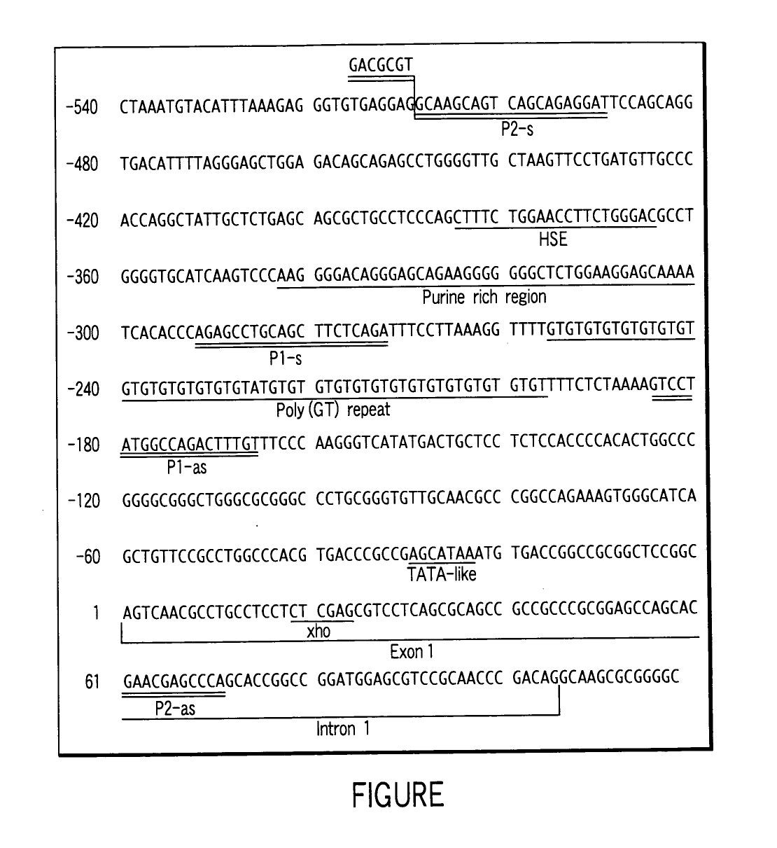

### CROSS-REFERENCE TO RELATED APPLICATIONS

This application is based upon and claims the benefit of priority from prior Japanese Patent Application No. 2003-168012, filed Jun. 12, 2003, the entire contents of which are incorporated herein by reference.

### BACKGROUND OF THE INVENTION

1. Field of the Invention

The present invention relates to a method for estimating the risk of incidence of lung cancer.

2. Description of the Related Art

Lung cancer is increasing in morbidity and mortality not only in Japan, but globally. There is an urgent need for methods for preventing lung cancer, the elucidation of the mechanism of lung cancer incidence including onset factors, development of diagnosing methods and the treatment methods thereof. Smoking is a major risk factor in lung cancer. During smoking, a variety of active oxygen species and carcinogens such as aromatic hydrocarbons and nitrosamines are inhaled. These constituents are believed to be related to DNA damage and lung cancer onset of respiratory tract epithelial cells. On the other hand, because only some smokers suffer from the incidence of these disorders, the involvement of intrinsic onset causes has been pointed out.

Most lung adenocarcinoma suppressor genes such as p53, which has been proved as a suppression gene, suppress the proliferation of lung cancer cells, and further have a function to kill lung cancer cells. It is understood that lack of this lung cancer suppressor gene causes lung cancer. On the contrary, the presence of anti-oxidizing enzymes such as gluthathione-s-transferase, which is an enzyme inactivating active oxygen species and carcinogens generated during smoking, is known. Enzymes with anti-oxidizing action have polymorphism, and the relationship between enzyme activity and chronic pulmonary emphysema and thus a smoking-related disease is being clarified.

For example, the present inventor has reported that heme oxygenase-1, which decomposes a heme to yield biliverdin, carbon monoxide and iron, restrains lung cell damage due to an oxidant. Furthermore, the inventor has shown that gene polymorphism, i.e. a long GT repeated sequence, hardly develops heme oxygenase-1 and associates with chronic pulmonary emphysema onset, which attacks a smoker (U.S. Pat. No. 6,436,645; Hirai H et al., the association between microsatellite polymorphism of heme oxygenase-1 gene promoter and sensitivity to oxidative injury in lymphoblastoid cell line, Eur. Respir. J., 2000, volume 16, p. 526).

Smoking, on the other hand, is believed to damage the DNA strands of the airway and alveolar epithelial cells to associate with lung cancer onset. An anti-oxidizing enzyme that suppresses DNA damage caused by smoking has been studied in terms of the relationship between a decrease in enzyme activity and lung cancer. However, so far there have been no evidence that the relationship between the activity of the anti-oxidizing enzyme and lung cancer incidence exist.

### BRIEF SUMMARY OF THE INVENTION

The present invention provides a method for evaluating an inclination of a subject to lung cancer, the method comprising preparing a DNA, from the sample, containing at least one allele which comprises a GT repeat located upstream to a heme oxygenase-1 gene; and determining the repeat number of the GT repeat in each allele in the sample wherein the presence of at least one allele comprising a GT repeat whose repeat number is not less than 33 being an indication for an inclination of a subject to lung cancer.

Furthermore, the present invention provides a kit for evaluating an inclination of a subject to lung cancer, the kit comprising a primer capable of amplifying sequences described in SEQ ID NO. 1.

Use of the method of the present invention can give data for evaluating an inclination of a subject to lung cancer. Accordingly, the result of this analysis can be used for a decision criterion for preventing lung cancer.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

The single FIGURE shows a primer used for DNA analysis and a amplified sequence.

### DETAILED DESCRIPTION OF THE INVENTION

The present invention has been made on the basis of epidemiological studies by the present inventors which have showed that polymorphism of GT repeat located upstream of the gene of heme oxygenase-1 (hereinafter, abbreviated as HO-1), which catalyzes the first reaction of heme metabolism, is associated with lung cancer incidence.

More specifically, the method of the invention evaluates an inclination of a subject to lung cancer by determining a repeat number(s) of a GT repeat(s) located upstream to an HO-1 gene.

The term “GT repeat” herein used means a repeated sequence in which repeat units consisting of a guanine(G) and a thymine(T) are repeated. The term “repeat number” herein used means the number of GT repeat units in a GT repeat.

First, to carry out the method of the invention, a sample containing a GT repeat located upstream of HO-1 gene is to be prepared. Such a sample may be any biological constituent sampled from a subject to whom the method of the invention is applied. The sample may be, for example, blood.

To “prepare a sample”, those who carry out the method of the invention may either prepare a sample for themselves or obtain a sample already prepared, thus a “samples” used for the present invention may be prepared in advance by a medical doctor, etc.

Preferable “subjects” to which the method of the invention are applied are mammals including human. However, polymorphism of GT repeat is known for many species other than mammals. Accordingly, any animal species can be a “subject” so long as it contains HO-1.

The method of the present invention, first, prepares a nucleic acid from the above-mentioned samples. A method of extracting nucleic acid from a biological component is well known to those skilled in the art, and any method may be used that includes, for example, phenol extraction or ethanol precipitation.

Following sample preparation, the repeat number(s) of a GT repeat(s) located upstream to an HO-1 gene is to be determined. Since the sequence of an upstream region of an HO-1 gene (Seq. ID No. 1) and a method for determining the repeat number of any repeat is well-known, those skilled in the art would easily determine the repeat number of a GT repeat.

In order to determine the repeat number of a repeat, polymerase chain reaction (hereinafter referred to as PCR) can preferably be used. To determine the repeat number of a GT repeat with PCR, the GT repeat is amplified by a primer pair consisting of unique sequences located upstream or downstream to GT repeat, the PCR product is applied to electrophoresis to determine mobility. Alternatively, in a case where a lot of samples are to be determined, DNA chip can be used to determine the repeat number of a GT repeat.

In a case DNA chip is used, a sample containing at least one allele which comprises a GT repeat is applied to DNA chip on which probes consisting of CA repeat unit are immobilized. Subsequently, the repeat number of the GT repeat may be determined by measuring a difference in denaturing conditions (e.g., melting temperature(Tₘ)) dependent on a difference in the repeat number.

For example, on condition that a DNA strand having a repeat of 33 times or more is hybridized and that a DNA strand having a repeat of less than 33 times is not hybridized, measurement of whether the DNA sample is hybridized with the probe or not can determine the repeat number of a GT repeat.

Furthermore, if necessary, the repeat number can be determined by directly sequencing a GT repeat.

As described in detail in an Example section below, subjects have a strong inclination to lung cancer, if they have at least one allele comprising a GT repeat which is located upstream to an HO-1 gene and its repeat number is not less than 33 lung cancer. Thus, it is possible to evaluate an inclination of a subject to lung cancer by determining the repeat number of a GT repeat. More specifically, an odds ratio of 2.2 was found by a series of epidemiological researches planned to investigate a correlation between an incidence of lung cancer and the repeat number of a GT repeat. An odds ratio of 2.2 means that if a subject has at least one allele which comprises a GT repeat whose repeat number is not less than 33, then he or she is exposed to a risk for lung cancer approximately 2.2 times higher than a subject who don't have such an allele.

In the method of the present invention, those who carry out the method are not limited to those who are engaged in activities of medical institutions.

In addition, the present invention provides a kit for evaluating an inclination of a subject to lung cancer by the above-described method. The kit includes primer capable of amplifying the sequence indicated in Seq. ID No. 1. For example, primers contained in a kit may include 5′-agagcctgcagcttctcaga-3′ (Seq. ID No. 2) and 5′-acaaagtctggccataggac-3′ (Seq. ID No. 3). Moreover, the kit may include, in addition to the primer, an enzyme required for conducting the PCR reaction, a buffer and a reagent for preparing DNA from the sample. In this case, the use of the primer can determine the repeat number by carrying out the PCR reaction. Additionally, use of the primer also allows the determination of the repeat number by determining the sequence of a GT repeated sequence.

Furthermore, the kit of the present invention may also include a probe having a repeat unit of CA, excluding a primer. In particular, the probe includes a DNA chip in which the probe is made a solid phase. In addition, the probe may include, besides a probe, a buffer for a hybridization reaction. Use of a probe contained in the kit of the present invention allows the repeat number to be determined by conducting the above-described method.

Hereinafter, the present invention will be set forth in detail by means of examples.

From 151 patients of lung cancer and 153 persons without lung cancer whose ratio of persons having smoking histories to nonsmokers is similar to that of the patients, peripheral vascular blood was sampled and its DNA was extracted. A PCR was carried out using the DNA as the template.

Primer pairs of a p1-s primer (nucleotides 249-268 of Seq. ID No. 1) and a p1-as primer (nucleotides 356-375 of Seq. ID No. 1), and a p2-s primer (GACGCGTGCAAGCAGTCAGCAGAGGAT) and a p2-as primer (nucleotides 591-611 of Seq. ID No. 1) were synthesized as primers for amplifying a GT repeat (nucleotides 285-344 of Seq. ID No. 1) located upstream to an HO-1 gene.

Then, the amplification by PCR was conducted using as the template genome DNA extracted in accordance with the normal method. The sizes of amplified products were analyzed by a laser base automatic DNA sequencer (Pharmacia, Uppsala, Sweden) using as primers fluorescence-labeled p1-s and unlabeled p1-as to determine the repeat number of a GT repeat.

The repeat numbers of a GT repeat had peaks at three places of 23, 30 and 33. Measured patients and control subjects were, according to the numbers of GT repeat, grouped into three classes: S being less than 27, M being 27 or more and less than 33, and L being 33 or more. Furthermore, the gene groups were divided into two groups: Group I (L/L, L/M, and L/S) and Group II (M/M, M/S, and S/S). As a result, the proportions of the number of GT sequences of Class L and the gene polymorphism of Group I are remarkably increased in lung adenocarcinoma patients (table. 1).

TABLE 1

| | | | Odds ratio | |
|---|---|---|---|---|
| | | Total number | (95% CI) | |
| | Number of | of lung | relative to | |
| | control | adenocarcinoma | all the other | |
| | subjects | patients | classes or | |
| | (N = 153) | (n = 151) | the subgroup | P |
| Allelic gene class | n = 306 | n = 302 | | |
| L | 39(13%) | 58(39%) | 1.6(1.0-2.5) | <0.03 |
| M | 137(45%) | 108(36%) | 0.7(0.5-1.0) | <0.03 |
| S | 130(42%) | 136(45%) | 1.1(0.8-1.5) | >0.5 |
| Inheritance | n = 153 | n = 151 | | |
| subgroup | | | | |
| I | 36(24%) | 54(36%) | 1.8(1.1-3.0) | <0.02 |
| II | 117(76%) | 97(64%) | | |

Now, according to the repeat number of a GT repeat, the total number of subjects containing both the smokers and the nonsmokers were classified into Class L (33 times or more), Class M (27 times or more and less than 33 times), and Class S (less than 27 times). As a consequence, the number of subjects in Class L, who have a large number of GT repeat, are substantially increased in lung adenocarcinoma patients. In addition, the subjects were classified, based on gene types, into Group I (L/L, L/M, and L/S) and Group II (M/M, M/S, and S/S). Consequently, the number of subjects in Group I, who have Class L with a large number of GT repeat, are substantially increased in lung adenocarcinoma patients (Table 1).

According to the number of GT repeat, the smoker subjects were placed into Class L (33 times or more), Class M (27 times or more and less than 33 times), and Class S (less than 27 times). Consequently, the number of subjects in Class L, who have a large number of GT repeat, are clearly increased in lung adenocarcinoma patients. Additionally, the subjects were fallen, according to gene types, into Group I (L/L, L/M, and L/S) and Group II (M/M, M/S, and S/S). As a result, the number of subjects in Group I, who have Class L with a large number of GT repeat, are substantially increased in lung adenocarcinoma patients (Table 2).

TABLE 2

| | | | Odds ratio | |
|---|---|---|---|---|
| | | Number of | (95% CI) | |
| | Number of | smoking lung | relative to | |
| | control | adenocarcinoma | all the other | |
| | subjects | patients | classes or | |
| | (N = 69) | (n = 73) | the subgroup | P |
| Allelic gene class | n = 138 | n = 146 | | |
| L | 15(11%) | 31(21%) | 2.2(1.1-4.3) | <0.02 |
| M | 74(54%) | 58(40%) | 0.6(0.4-0.9) | <0.02 |
| S | 49(35%) | 57(39%) | 1.2(0.7-1.9) | >0.1 |
| Inheritance | n = 69 | n = 73 | | |
| subgroup | | | | |
| I | 15(22%) | 30(41%) | 2.5(1.2-5.2) | <0.02 |
| II | 54(78%) | 43(59%) | | |

According to the number of times of GT repeated sequence, the nonsmoker subjects were classified into Class L (33 times or more), Class M (27 times or more and less than 33 times), and Class S (less than 27 times). The number of subjects of Class L having a large number of GT repeat shows no difference between the lung adenocarcinoma patients and the control subjects. In addition, the subjects are classified, according to gene types, into Group I (L/L, L/M, and L/S), and Group II (M/M, M/S, and S/S). As a consequence, the number of subjects of Group I having Class L with a large number of GT repeat indicates no difference between the lung adenocarcinoma patients and the control subjects (Table 3).

TABLE 3

| | | Number of | Odds ratio | |
|---|---|---|---|---|
| | | nonsmoking | (95% CI) | |
| | Number of | lung | relative to | |
| | control | adenocarcinoma | all the other | |
| | subjects | patients | classes or | |
| | (N = 84) | (n = 78) | the subgroup | P |
| Allelic gene class | n = 168 | n = 156 | | |
| L | 24(14%) | 27(17%) | 1.3(0.7-2.3) | 0.5 |
| M | 63(38%) | 50(32%) | 0.8(0.5-1.2) | 0.3 |
| S | 81(48%) | 79(51%) | 1.1(0.7-1.7) | 0.7 |
| Inheritance subgroup | n = 84 | n = 78 | | |
| I | 21(25%) | 24(31%) | 1.3(0.7-2.7) | 0.4 |
| II | 63(75%) | 54(69%) | | |

From the above-described results, a gene polymorph having a large number of GT repeat in a heme oxygenase-1 gene is associated with lung adenocarcinoma incidence of a smoker (table 2). Additionally, nonsmokers show no difference in the number ratio of Class L and Group I between the lung adenocarcinoma patients and the non-lung adenocarcinoma control subjects (Table 3). From this result, it has been found that a gene polymorphism having a large number of GT repeat of a heme oxygenase-1 gene is associated with the incidence of lung adenocarcinoma in a smoker.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A method for evaluating an inclination of a subject to lung cancer, comprising:
(a) preparing a DNA, from the sample, containing at least one allele which comprises a GT repeat located upstream to a heme oxygenase-1 gene; and
(b) determining the repeat number of the GT repeat in each allele in the sample wherein the presence of at least one allele comprising a GT repeat whose repeat number is not less than 33 being an indication for an inclination of a subject to lung cancer.

2. The method according to claim 1, wherein the sample is blood sampled from the subject.

3. The method according to claim 1, wherein the repeat number of the GT repeat in (b) is determined by applying PCR-amplified GT repeats to an electrophoresis.

4. A kit for evaluating an inclination of a subject to lung cancer according to the method of claim 1, comprising:
a primer capable of amplifying a sequence indicated in Seq. ID No. 1.

5. The kit for evaluating an inclination of a subject to lung cancer according to claim 4, further comprising:
a probe having a repeat unit of CA.
